(19) United States
(12) Patent Application Publication
(10) Pub. No.: US 2023/0197289 A1
(43) Pub. Date: Jun. 22, 2023

(54) Epidemic Monitoring System

(71) Applicants: Laurence Richard Olivier, Alpharetta, GA (US); LifeQ B.V., Amsterdam (NL)

(72) Inventors: Tom deLaubenfels, Atlanta, GA (US); Franco du Preez, Cumming, GA (US); Etienne Terblanche, Alpharetta, GA (US); Laurence Richard Olivier, Alpharetta, GA (US)

(21) Appl. No.: 17/922,394

(22) PCT Filed: Apr. 29, 2021
(86) PCT No.: PCT/US2021/029940

§ 371 Date: Oct. 31, 2022

Related U.S. Application Data
(60) Provisional application No. 63/017,254, filed on Apr. 29, 2020.

(51) Int. Cl. G16H 50/80; G16H 50/30; G16H 40/67; G16H 50/70

(52) U.S. Cl. CPC G16H 50/80; G16H 40/67; G16H 50/30; G16H 50/70

(57) ABSTRACT

Systems and methods for monitoring the spread of pandemic pneumonia using IOT technology is provided. Sensor data from wearable devices is utilized to determine: a probability of developing complications from a pandemic for an unexposed user using existing indicators of the wearer’s health; the impact of lockdown measures on health; a probability that a user exposed to the pathogen experiences complications; and a probability of various disease stages for the user including normal, asymptomatic, pre-symptomatic, symptomatic, complication development and recovery is provided.

### FIELD OF THE INVENTION

The invention relates to the tracking of transmission, disease stages, vulnerability to complications and impact of regulations to control the spread of a novel pathogen using location and physiological data gathered by mobile sensors including wearables and GPS.

### BACKGROUND OF THE INVENTION

Many government agencies across the planet seek to track the presence, spreading and impact of novel diseases in order to mount an appropriate response in time. Without exception, there is currently global difficulty in quantifying the spread of disease that possesses pandemic potential. For example, tracking the basic progression of a highly infectious disease through a population, via contact tracing and other typical methods, is a labor intensive process requiring significant human resources and comprehensive logistical systems. As another example, successful disease tracking efforts require new infections to be discovered and recorded in as short a time as possible, either by direct confirmation from clinical-standard tests or by the presumption of infection based on the occurrence of applicable symptoms; this problem also requires significant human and logistical resources within standard medical and public health systems. The aforementioned issues become increasingly problematic in countries/jurisdictions which are resource-poor.

The problems described above pertains to the benefit of government bodies in charge of epidemic management or otherwise. There are however also significant problems that individuals face in a pandemic that is not solved by government or company policy adjustments or other interventions actioned by the bodies. For example, there are smaller bodies that are responsible for the health of a group of people, like an employer. For an individual that is being monitored in an epidemic, an important problem is to know when she becomes infected, so that she prevents family members, coworkers and other individuals from contracting the disease. It is also important for her to know which of her family or loved ones are infected in order to provide support and prevent further spreading of the infection among these individuals. Furthermore, it is important for her to be able to monitor the health status of individuals that need to access her living space, for example a caretaker for a parent residing at the family home. When she or family, remote or in the same living quarters, do become infected, it is important to be able to follow their level of health throughout the infection and to receive alerts when there are signs of dangerous complications developing, such that she can provide the necessary support to these individuals. Beyond this, it is also important for her to know the risk of severe complications for family and loved ones, based on their medical history, demographics and other health indicators ahead of them contracting disease, to help regulate the level of exposure levels of individual family members. Finally, another problem that the monitored individual faces, is how to control her behaviour to lower the risk of spreading infection to the population at large and similarly to help inform friends and family as to the appropriate levels of caution in her area.

Therefore, there is a need for a solution that addresses the problems mentioned above.

### SUMMARY OF THE INVENTION

The proposed solution, which will be described in detail forthwith, adds an approach leverages accessible consumer electronic devices and established IOT technologies known to those skilled in the art, in conjunction with learnings in domains of clinical care and epidemiology research which are continuously being improved. As an example, current estimates show that 20% of US adults make use of wearable devices, which is a significantly greater portion compared to the small fraction of individuals entering the healthcare system on a day to day basis during a pandemic. Similar to the use of Amber alerts on mobile phones, which lead to a coordinated community wide response to alerts, wearable and other IOT body monitoring devices can be regulated to enable such functionality under the conditions of a pandemic. This provides a substantial advantage over the fragmentation over different healthcare systems in different states. Furthermore, whereas information transfer from the aggregate of different healthcare and emergency systems to government institutions lead to a substantial delay in information transfer, the proposed solution can greatly augment the speed of information transfer. These methods are sufficiently flexible to be applicable to both the US and other countries/jurisdictions, including those which are comparatively resource-poor.

One important consequence of the low coverage of individuals through the relaying of pandemic information through the healthcare and emergency services is that the low density of information on early symptoms prevents high resolutions geographic maps to be constructed for helping to locate the origin and contacts for new cases. With a high coverage IOT system, wearable and mobile devices enable pinpoint GPS coordinates associated with changes in physiology that can be used to track the early stages of an epidemic in high resolution allowing early action to suppress it.

In an aspect, the invention is directed at a method for providing continuous monitoring of a population for infectious disease that includes the steps of detecting anomaly associated deviations in IOT data-streams for a monitored individual and screening the deviations through communication with the monitored individual to predict whether they were caused by confounding factors instead of a disease. The anomaly associated deviations can include deviations in physiological and behavioral datastreams, deviations derived from a comparison between population data and current data of the individual, and/or deviations derived from a comparison between historical data of the individual and current data of the individual. Screening the deviations can include assessing factors including, but not limited to, alcohol consumption, heavy meals close to bedtime, strenuous exercise, and/or psychological stress.

In an aspect, the method can contact the monitored individual to report the prediction of disease, including informing the monitored individual of possible infection and a risk of developing complications. The method can monitor several individuals, and combine anomaly associated deviations in a discreet disease state space to provide predictions on the disease status and risk of developing severe complications in case of infection for each monitored individual in order to control physical access to a work site, discover new epidemics or new disease outbreak hotspots in existing pandemics, and/or perform contact tracing to warn individuals of potential exposure. The contact tracing can be done over the location history of multiple users and sends notifications through a communication module to users that might have been exposed to a user with a confirmed or suspected infection. Historic location data used for contact tracing can be routed through likely establishments visited by users to compute the likelihood of local contact between users using meta-information on establishments, exemplified by having a low contact likelihood for an inferred gas station visit for two users compared to an inferred visit to a gym. In addition, the historic pattern of inferred disease state transitions can be used to forecast future patterns of disease states.

In an aspect, after screening the deviations to confirm causes by a disease, the anomaly associated deviations may be used to predict transitions in a discreet disease state space for a monitored individual using a quantitative model to predict transitions. In an embodiment, the discrete disease state space may include general health to describe general disease. The level of general health of the user is determined to serve as a proxy for the risk of contracting severe complications during the disease. This is done considering a subset of the following group of measures: heart health (through pulse waveform analysis, heart rate variability measures, and heart rate measures), respiratory health (through SpO2 and breathing rate), activity and fitness levels (through actigraphy and heart rate reserve changes, sleep health (through total sleep time, duration of sleep stages, continuity of sleep stages, and sleep quality estimates from the preceding).

In other embodiments, the discrete disease state space can include multiple disease states to represent alternative diseases or disease groups. In an aspect, the quantitative model is used to predict transitions in the discrete disease state space based upon said anomaly associated deviations, the IOT data streams of the monitored individual (including measured behavior, estimated behavior, measured physiology, and/or estimated physiology), and feedback related to the monitored individual, the feedback including symptoms, disease status based on clinical test results, exposure, and/or confounding factors, including alcohol consumption, heavy meals close to bedtime, strenuous exercise, and/or physiological stress.

In an aspect, the quantitative model can utilize epidemiological parameters known for diseases to make transitions in the discrete disease state. The epidemiological parameters of the disease can include incubation time, symptomatic disease duration, a R0 value of the disease, expected physiological deviations, expected behavioral deviations, or geospatial and temporal coordinates of the monitored individual and publically available estimates of disease prevalence at the location. The quantitative model can also be trained on population data including documented cases of disease and time of transitions in the disease state space.

In an aspect, the present invention is directed at a method of continuous monitoring of a population for infectious disease, the method including capturing, from mobile devices associated with individuals, physiological signals associated with the individuals, time information, and location information related to the mobile device, comparing the captured physiological signals against historic physiological signals associated with the individuals to identify anomalies, calculating anomaly associated deviations from the identified anomalies, clustering the anomaly associated deviations into similar groups at a population level, selecting anomaly groups based on infectious disease knowledge, screening the anomaly associated deviations of the selected anomaly groups on spatiotemporal correlation over the population level, and communicating potential epidemic findings if the spatiotemporal correlation meets threshold. In an aspect, the method can also include flagging the anomaly associated deviations based on infectious disease knowledge after calculating anomaly associated deviations.

In an aspect, the invention is directed at a system for providing continuous monitoring of a population for infectious disease. The system includes a server with a network connection that allows communication with a plurality of IOT devices associated with a plurality of individuals. The server includes memory and processor that calls upon a disease stating module, an anomaly detecting module, an anomaly screening module, a probability assigning module, and a general communicable disease state space. The server receives physiological data streams of the plurality of individuals via the network connection from the plurality of IOT devices. The processor can call upon the anomaly detecting module to detect anomalies in physiological data and the probability assigning module to assign probabilities to the states in the disease state-space model based on the anomalies that pass the screening The physiological data found in the physiological data streams is recorded from physiological systems, including the cardiovascular, pulmonary, musculoskeletal, and neurological systems. The data of that data streams can include heart health (pulse waveform analysis, heart rate variability measures, heart rate measures), respiratory health (SpO2, breathing rate), activity and fitness (actigraphy and heart rate reserve changes), and sleep health (total sleep time, duration and continuity of sleep stages, and the quality of sleep).

In an aspect, the system can screen the anomalies based on pre-existing knowledge of general changes in physiological signals for communicable disease and/or similarity in the nature and timing of physiological deviations in a subgroup of the monitored population. In an aspect, the system includes a timing module to gather information on the timing of anomalous events across multiple users. The system can also include a location module configured to gather information on the proximity of the users of the system to one another. The system, correlating the location and time information via the disease stating module, can filter for anomalies with a shared physiological deviation pattern among a subgroup of the monitored population. The disease stating module can also be configured to personalize over time using physiological data from the respective individual. In an aspect, the system can include a communication module that can notify a user and collect questionnaire responses from a user, which can also take answers to the questionnaires to label physiological data as disease associated anomalies supporting a transition in the disease state space. The questionnaire data can include subjective indications of wellbeing exemplified by chills, fatigue and general malaise, as well as data that covers exposure risk information, including but not limited to local shelter in palace orders, family size and occupation type and status. The disease state module can be personalized on a per individual basis with the labelled data.

In an aspect, the server is configured to communicate data from various IOT devices with similar sensing capability, enabling the collection of normalized, cross device compatible physiological signals. In another aspect, the anomaly detection module makes use of a deep learning architecture that captures a low dimensional latent space representation of a user’s physiological signals. The shared latent space dimensions (as defined for VAE or GAN approaches mentioned) can be used to compare physiology across multiple individuals. In an aspect, the anomaly detection module’s latent space has been constructed such that the latent space dimensions of one device architecture recording a particular physiological signal, can be related to that of another device recording the same physiological signal.

In an aspect, the anomaly detection module of the system is configured to gather anomalies across a population of users to filter out deviations that are not common to a subgroup of users that are temporally correlated. The anomaly screening module can also filter for development of pneumonia by using decreases in an SpO2 physiological signal or increases in breathing rate signal to filter for anomalies indicative of the development of pneumonia. The anomaly screening module can use changes in heart rate to filter for anomalies indicative of communicable disease onset or progression. Further, the anomaly screening module can filter anomalies for a presymptomatic state in the disease state space according to raised resting heart rate, lowered HRV, and/or lowered activity. In an aspect, the anomaly screening module can filter anomalies for a symptomatic state in the disease state space according to raised HR, lowered HRV, lowered activity and fitness, and/or Arrhythmias patterns. In another aspect, the anomaly screening module can filter anomalies for a complication development state in the disease state space according to patterns in: decreased SpO2 (indicating pneumonia progression), specifically increased breathing rate for pneumonia, using oxygen therapy (bronchodilators, focused monitoring early to improve outcome), and arrhythmias (indicating cardiac complication progression). In an aspect, the anomaly screening module can filter anomalies for a recovery state in the disease state space according to patterns in increased resting heart rate, increased HRV, and increased activity.

In an aspect, the location module of the system determines the proximity of users by GPS data using GPS capabilities in a wearable device, or other mobile device with such capacity capable of networking with it, such as a mobile phone. The location module can also rely on coarse user proximity data by using meta information associated with the wearable data such as the server from which the data is received, for example the country from which the data is sent. The location module can also rely on user proximity data by using local networking information, exemplified but not limited to positioning via Wifi or Bluetooth signals.

In an aspect, the system can process data and the algorithmic discovery of anomalous events takes place on a physiological data recording device, a companion device such as a smartphone, a cloud server or any other appropriate networked device depending on energy, processing and storage constraints. In an aspect, the physiological information includes auditory data relevant to coughing, heart sounds and breathing rate through appropriate mobile sensor technology exemplified by a MEMS microphone enabled smart patch. The physiological information can include data on the cardiovascular system exemplified by PPG-derived heart rate, heart rate variability and SpO2.

In an aspect, the disease state utilized by the system includes a presymptomatic state where the user can be alerted to possible infection prior to experiencing symptoms. The disease state space can include an asymptomatic state where the user can be alerted to possible infection without experiencing symptoms through the course of the disease. The disease state space can include a symptomatic stage where the user can be alerted to possible infection and where this can be confirmed or denied based on medical test results shared by the user. The disease state space can include a complication state where the user can be alerted to the development of possible complications, exemplified by pneumonia due to anomalously low SpO2 readings from a mobile monitoring device. The disease state space can include a recovery state where the user can be alerted to changes in physiological signals indicative of either a worsening or an improvement in physiological readings and where a user is alerted to physiological readings that no longer deviate significantly from readings collected during a healthy state.

In an aspect, the system includes an epidemiology module that aggregates over a population of users, information on disease states from the disease state module, anomalies with their associated deviations in physiological signals and their timing. The epidemiology module can predict whether there is evidence for an epidemic in the population that is being monitored based on a shared pattern of physiological deviations across multiple users where physiological deviations of similar nature are spatiotemporally correlated and are evolving spatiotemporally in a manner that fits a quantitative epidemiological model, exemplified by exponential growth in number of predicted symptomatic cases in time at correlated geographic locations.

These and other objects and advantages of the invention will become apparent from the following detailed description of the preferred embodiment of the invention. Both the foregoing general description and the following detailed description are exemplary and explanatory only and are intended to provide further explanation of the invention as claimed.

The accompanying drawings are included to provide a further understanding of the invention and are incorporated in and constitute part of this specification, as well as illustrate several embodiments of the invention that together with the description serve to explain the principles of the invention.

### BRIEF DESCRIPTION OF THE DRAWINGS

FIG. 1 is a block diagram indicating the different electronic devices that make up the system

FIG. 2 is a series of operations outlining how the components of the system can be applied

FIG. 3 is a block diagram illustrating the system for discovering and monitoring epidemics in a population of users wearing devices connected to the internet and capable of recording physiological and temporal information. Calculations to support this function take place predominantly on a processor that is connected to the internet and rather than part of the body wide network of the end user.

FIG. 4 is the left half of a block diagram (right half in FIG. 5) illustrating the system for discovering and monitoring epidemics in a population of users wearing devices connected to the internet and capable of recording physiological and temporal information. In this embodiment, emphasis is placed on performing computation on devices in the user’s body wide network.

FIG. 5 is the right half of a block diagram (left half in FIG. 4) illustrating the system for discovering and monitoring epidemics in a population of users wearing devices connected to the internet and capable of recording physiological and temporal information. In this embodiment, emphasis is placed on performing computation on devices in the user’s body wide network.

FIG. 6 shows a disease staging model for Covid-19 with all possible states that constitutes the disease state space, as well as the knowledge based screening rules in italics.

DEFINITIONS

| | |
|---|---|
| BAN | Body area network |
| ECG | Electrocardiogram |
| GAN | Generative Adversarial Network |
| HMM | Hidden Markov Model |
| HRV | Heart Rate Variability |
| ICU | Intensive care unit |
| IOT | Internet of things |
| PPG | photopiethysmography |
| SpO2 | percentage of oxygen saturated hemoglobin |
| VAE | variational auto-encoder |

### DETAILED DESCRIPTION

The invention presented provides a solution to the problem of automated discovery and monitoring of epidemics using mobile technology to gather physiological signals across multiple mobile devices. Human pathogens are a cause for epidemics, through the human disease states that they create. For example, bubonic plague is a disease caused by a bacterial pathogen, Yersinia pestis and it caused the death of many millions between 1346 and 1353 AD. Communicable disease is caused by a transmittable human pathogen and can be contrasted against non-communicable diseases. Epidemics are not necessarily caused by communicable diseases, but can also be non-communicable, for example the obesity epidemic, where changes to the human diet and lifestyle has resulted in a rapid increase in the number of humans that are overweight to the point of being unhealthy.

The importance of monitoring and early detection of epidemics can be understood considering that there are many actions that can be taken by either individuals or organizations and governments to drastically change the course of an epidemic. The recent Covid-19 outbreak has been the cause for applying aggressive measures to limit citizen mobility across many countries globally, but the detrimental impact on economies, including job losses, highlight the importance of applying these measures as precisely and measured as possible to balance containment of the virus against the economic downturn and reduction in general health due to lockdown regulations. High geospatial and temporal resolution information on symptoms, disease onset and progression derived from wearable data during such a pandemic can help to balance decisions impacting millions of lives and trillions of dollars. This includes not only identifying symptomatic cases, but also performing contact tracing to manage pre-symptomatic cases.

While very early detection and contact tracing in a monitored population can lead to novel pathogens being successfully contained, this becomes increasingly improbable as more become infected This requires not only reactive measures to monitor a recognized epidemic but also proactive measures. The disclosed invention, which will be described in detail forthwith, is intended to serve as an applicable proactive measure when operating at large scale. Novel pathogens by definition require new processes for establishing a diagnosis and diagnosing the first case requires time consuming research while the epidemic can reach an irreversible state. Automatically recognizing common physiological deviations from baseline in a monitored population, where these deviations are also seen at correlated geographic locations and time points, can help to rapidly establish focus on a potential new epidemic and the individuals affected.

IOT monitors of human physiology are in widespread and increasing use. At the time of writing, one in five Americans are using such a device. The computing capabilities of these devices has also increased substantially with smartwatches having powerful processing capabilities relative to the fitness trackers that they are replacing. A key problem to solve that we address in the present invention, is to combine the analysis of data recorded for the same physiological signal across multiple devices of disparate design. In an aspect, the present invention can leverage the use of universal software libraries for such devices (e.g., google wear OS or similar).

Independent of a new epidemic, the pre-existing dimensions of health of an individual can be quantified through the collection of longitudinal physiological data. Longitudinal data refers to any pertinent measurements, signals, or other information types which are recorded with suitable frequency, and over sufficiently extended (or indefinite) durations, such that natural variations in the physiology of individuals being monitored can be separated from variations that are indicative of disease onset and recovery, e.g. on the scale of weeks to months (or longer). With Covid-19, cardiovascular, respiratory and immune system health are known to strongly influence the probability of complication development once an individual contracts the virus. We present as part of the system a solution to quantifying aspects of health relevant to predicting the risk for an individual of developing complications should she contract an infection. Said aspects would typically include demographic information such as height, weight, age and gender as well as health record data, including pre-existing conditions, but might also include data obtained through existing IOT monitoring through wearables, for example, a person’s resting heart rate value. There is clearly utility in having information on exposure risk handy for individuals, their friends, family and colleagues as this information can help the individual and community to most effectively protect those at the highest level of risk.

Aside from understanding the infection status and risk for complication development, recognizing signs of complication development in real time is of high importance for some diseases. In the case of Covid-19 for example, many patients arrive at medical institutions for unrelated reasons such as trauma, only for clinicians to discover that they suffer from advanced pneumonia and exhibit decreased oxygen saturation (SpO2) and increased breathing rate without them being aware of having pneumonia. The current generation of wearable technology increasingly includes the necessary technology, in the form of red and infrared light and sensors to get predictions of SpO2, which are still of dubious quality when taken on the wrist. Typical commercial wearable devices containing both photoplethysmography (PPG) and tri-axial accelerometer sensors are suitable for detecting/indicating the presence of disease infection, e.g. Covid-19, via symptomatic changes in vital signs, which can be tracked through these sensors using digital signal processing and machine learning approaches. We disclose here a method for making use of these technologies to fundamentally contribute to tracking the progression of infections as well as warning of signs of complication development. In resource-poor settings or overwhelmed medical systems, such capabilities can be of high value when round-the-clock monitoring in ICU beds becomes fully occupied.

During an epidemic, public health levels suffer not only due to the disease caused by the pathogen involved, but also the lock-down regulations in place to control transmission. Measuring health level decline under these conditions can help to provide valuable information on the wider impact of regulations and can help to balance between containing transmission and maintaining overall health levels. Utilizing already established wearable device networks can help measure health levels during lock-downs. This can be achieved through, for example, government mandated or opt-in mechanisms for loading firmware updates on devices to enable transfer of standardized algorithms and data transmission protocols. This could be analogous to the universal Amber Alert, capability supported by a wide variety of mobile phones. It might also be done at an employer level for example to better help enable risk management within a company where employees need to be in close proximity. It is also useful to monitor the general health trajectory of individuals as they recover from an infection, in order to help inform choices. Monitoring of the recovery trajectory can be done by accessing the individual’s physiological data via the aforementioned methods and assessing their relevant vital signs using digital signal processing and machine learning approaches. By using these same techniques and technological infrastructure, the system presented here can also help solve the problem of efficiently monitoring epidemics on the population scale.

For an individual being monitored within the system, the benefits can be manifested through direct interaction with a computer or mobile device connected to the internet, by which the individual can receive notifications such as changes in their predicted disease status, or requests for additional information/feedback which can help better establish, for example, a transition from an uninfected to infected state. Additional information/feedback requested of a monitored individual within the system may be collected, for example, via questionnaires pertaining to current symptoms and/or known instances of exposure to infected individuals; this can be done using suitable IOT enabled devices such as the individual’s own smart phone or personal computer, or alternatively through the user interface of a suitable smart watch or other worn device, through software applications, web forms, email surveys, etc., which are part of the disclosed system. Additionally, a user might also receive information not only about their own data, but also about anonymized findings from other users, globally or in their more immediate environment, as well as how their results compare to other relevant subsets of users. Beyond the benefits of direct feedback to a monitored individual through the sharing of information, consented third parties, such as employers or government organizations can also act to the benefit of the monitored individual by, for example, changing office policy or local shelter in place regulations respectively.

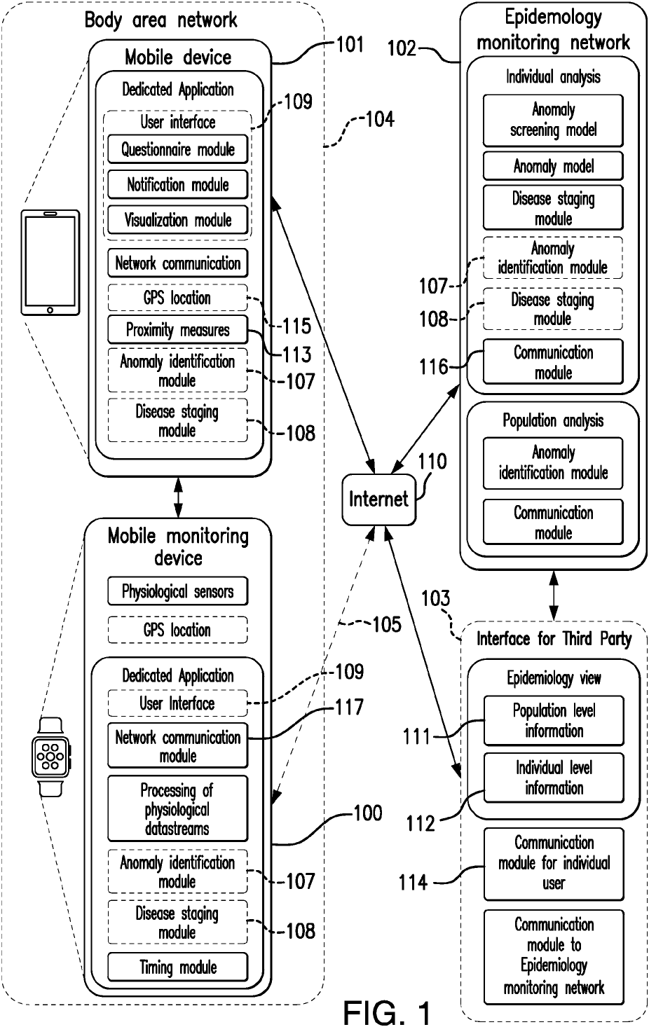

The basic constituents of the system proposed to discover and monitor novel epidemics are shown in FIG. 1. In an embodiment, the system consists of a number of IOT devices, including a mobile phone 101, a wearable device, termed Mobile Monitoring Device 100, which is capable of collecting timestamped physiological data from sensors such as, but not limited to, PPG and accelerometers, and transmitting said physiological data off of the local device; a server, or combination of servers, hosting the Epidemiology Monitoring Network 102 of the system, which receives physiological data transmitted from monitoring device(s) 100, either via direct internet connection 105 or through the mobile phone 101 as a proxy. In the latter case, the Mobile Monitoring Device 100 must be enabled with alternative means to transmit data locally to the mobile phone 101, either through physical wired connections or via wireless methods such as, but not limited to, communication through local area networks or protocols such as Bluetooth. Optionally, the system also provides an interface for consented third parties 103 to see the population level results, potentially overlaid on a map in instances where individual mobile monitoring devices 100 and/or mobile phones 101 support the collection of geospatial data, e.g. through GPS or location-specific network connectivity. When the Mobile Monitoring Device 100 is a smart watch that has both a direct internet connection 105 and suitable user interface, it is also possible to move the entire functionality of the mobile phone 101 to the Mobile Monitoring Device 100, and to omit the mobile phone 101 system entirely.

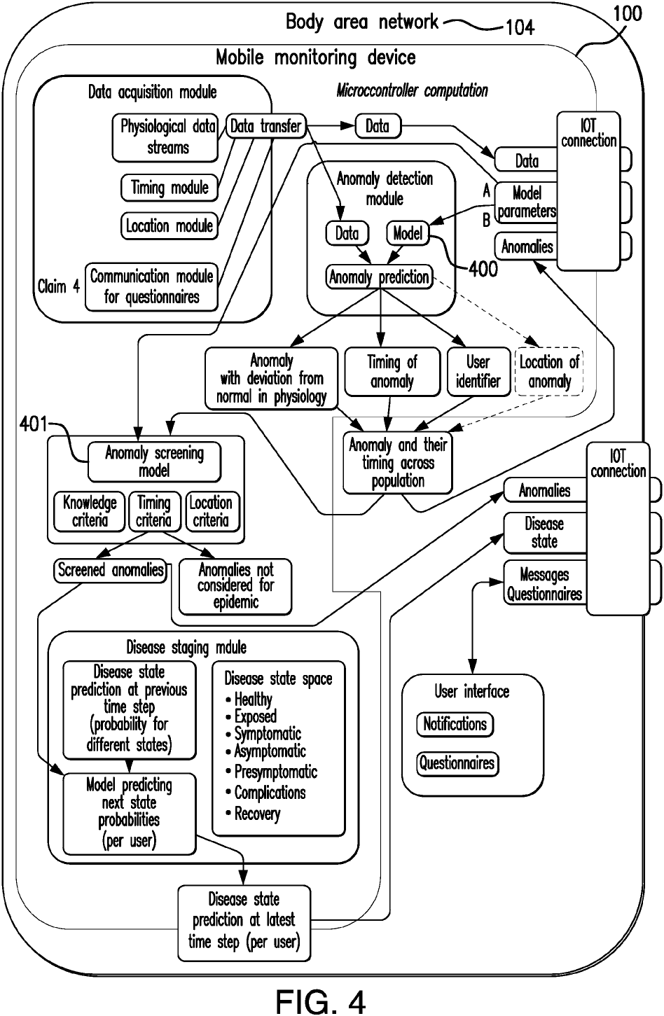

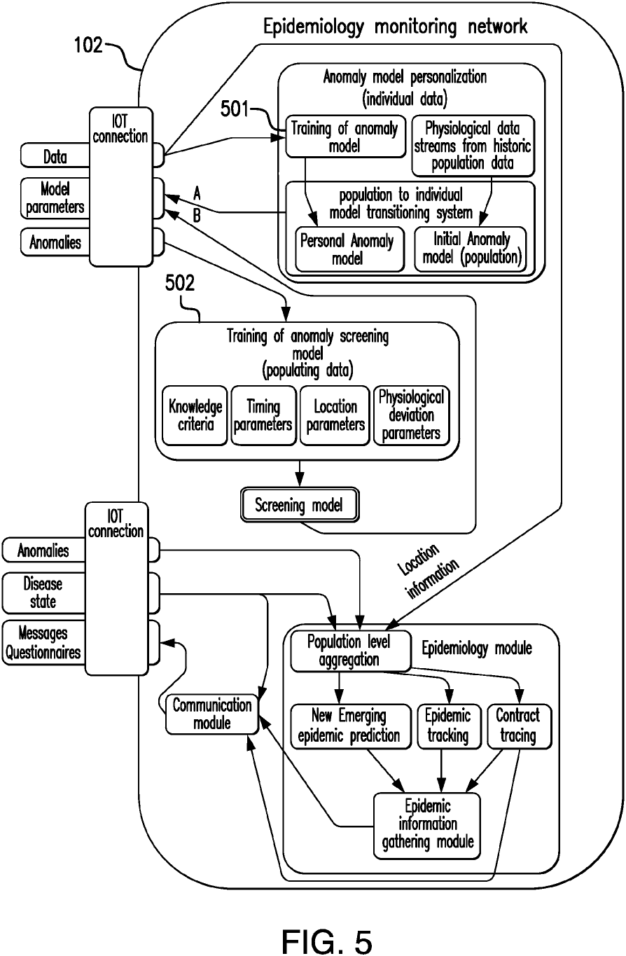

In FIG. 1, the computational tasks of processing physiological data, finding anomalies, and identifying possible transitions into disease or recovery states are indicated with dashed outlines to show that these modules can reside either in the Mobile Device 101, Mobile Monitoring Device 100, or alternatively in the Epidemiology Monitoring Network 102. These alternative embodiments are also described in more detail in FIG. 4 and FIG. 5. Similarly, in one embodiment, the user interface for an individual using the system on the Body Area Network (BAN) 104 (shown in FIG. 1) can be located on the Mobile Monitoring Device 100, or alternatively on the Mobile Device 101. The BAN 104 may contain multiple IOT devices, including mobile monitoring devices 100, which may be devices other than commercial wearable monitors e.g. smart watches. Examples of devices compatible with the BAN 104 include, but are not limited to: wearable devices other than commercial smart watches, such as patches containing various physiological sensors (PPG, electrocardiography, temperature, electrodermal, etc.), nearable devices e.g. video cameras or photodetectors in a monitored individual’s immediate environment, which are capable of producing physiological measurements such as remote PPG or temperature sensing; and /or ingestible sensors. In order to be compatible with the BAN 104, it is desirable that the sensor-containing device be capable of collecting and transmitting timestamped physiological data from the monitored individual to embodiments of the Epidemiology Monitoring Network 102, either via direct internet connection 105 or through a mobile phone 101 as a proxy. In the latter case, the Mobile Monitoring Device 100 must be enabled with alternative means to transmit data locally to the mobile phone 101, either through physical wired connections or via wireless methods such as, but not limited to, communication through local area networks or protocols such as Bluetooth.

In terms of more specific embodiments of the devices in the BAN 104, the Mobile Monitoring Device 100, can take many forms considering only present technologies in the market. At a minimum, such a device has the capability of recording a physiological signal and either processing it or sending it directly via a network 105, or through proxy devices in the BAN 104 to the internet. In certain embodiments, examples of the Mobile Monitoring Device 100 can include wrist worn wearable devices, such as smartwatches and fitness trackers that have PPG capabilities as well as accelerometers to measure actigraphy. Using data from the PPG and actigraphy, data can be extracted on human physiology and behaviour and this can be further processed to estimate the occurrence of behaviours which can be modified by the presence of infection, such as exercise and sleep, as well as the values of vital signs, such as heart rate and breathing rate, via the modulating effect that it has on individual heartbeats Many other physiological and behavioral measurements relevant to the presence of infection, and/or the recovery from infection, can be derived from compatible IOT monitors of human physiology. Examples include, but are not limited to, various indices of heart rate variability, cumulative levels of measured actigraphy throughout waking periods, deviations from normal routines established by longitudinal monitoring e.g. regular travel to/from work, number of steps taken per day, etc. In another set of embodiments, the Mobile Monitoring Device 100 can be exemplified by wearable patches or other devices capable of reading chemical markers through the skin, such as glucose monitoring patches. In yet another embodiment, smart eyewear with PPG capability can serve as the Mobile Monitoring Device 100, from which heart rate, indices of heart rate variability, and various associated insights (e.g. sleep and exercise periods) may be measured or inferred. In yet another embodiment, smart earphones with PPG capability can serve as the Mobile Monitoring Device 100. In yet another embodiment, mobile electrocardiogram (ECG) monitors, available in for example a patch form factor positioned on the chest can serve as the Mobile Monitoring Device 100. In other embodiments, such wearable patches can make use of auscultation to record heart and breathing sounds and serve as the Mobile Monitoring Device 100. In yet another embodiment, ingestible physiological monitoring devices such as smart capsules can serve as the Mobile Monitoring Device 100. Additionally, the Mobile Monitoring Device 100 can also be a nearable device, instead of a device worn on the body. For example, a nearable can include a camera, which allows for remote PPG signal generation, or a weight scale, which can record weight and other measurements known to be captured by such devices (body fat percentage, hydration levels, etc.). In another such embodiment the Mobile Monitoring Device 100 is a smart steering wheel with touch sensors that can measure accessible physiological signals exemplified by ECG, PPG and bioimpedance measurements. The overall system disclosed does not imply that a single Mobile Monitoring Device 100 should necessarily be used in all embodiments of the invention, with more devices covering a wider space of relevant physiological signals being favored over single devices or single physiological signals. In an aspect, the system is configured to utilize any IOT device capable of capturing some physiological data related to an individual.

The Mobile Device 101 is an optional companion to the Mobile Monitoring Device 100 described above and can be a smartphone or laptop. In certain embodiments, the Mobile Device 101 serves primarily to relay data and information between the Epidemiology Monitoring Network 102 and the mobile monitoring device 101, and in other embodiments to perform processing of the data from the Mobile Monitoring Device 100. In some embodiments, the Mobile Device 101 may also collect additional information/feedback from monitored individuals within the system, which can help better establish, for example, transitions from an uninfected to infected state; this information/feedback may be directly requested from a monitored individual by the Epidemiology Monitoring Network 102, for example, via questionnaires pertaining to current symptoms and/or known instances of exposure to infected individuals. Information/feedback requested from monitored individuals may be received through software applications, web forms, email surveys, etc., which operate on the Mobile Device 101 as part of the disclosed system. In other embodiments, the Mobile Device 101 can also be one or more stationary devices capable of communicating with the internet, exemplified by network routers that can communicate with both the Mobile Device 101 and the internet 105. The proximity measures component 113 on the Mobile Device 101 includes a range of methods to determine the proximity of users to one another through means other than GPS, such as Bluetooth connectivity, Near-Field Communication (NFC) and Wi-Fi connectivity for the user or multiple users making use of the system in some embodiments and can help to localize the user while inside buildings, where GPS signal is compromised, but also to link to the specific establishments that might be visited inside a large building. Knowing that an individual has been in the proximity of another user in this case is an essential piece of information to perform contact tracing as part of managing an epidemic. The resolution of proximity detection may be high, e.g. capable of measuring proximity on the order of meters, or it may be relatively lower, e.g. capable of determining that two individuals were in the same building at the same time; the former is preferable from the perspective of public contact tracing and establishing exposure risk, but the latter is still of merit and can help inform decisions on the part of the monitored individual, consented third parties, and/or public health authorities. Similarly, knowing the timing of when a monitored person passed a smart object can provide valuable data on areas, like buildings that might now be considered exposed if the person happens to harbor an infection predicted by the system, or otherwise known to the system via consented access to medical patient data. In addition to such local information, this module can also be used to embed information such as the country that the Mobile Device 101 was in, which can still be of use aside from high resolution GPS data. In other embodiments, the location information can also be requested and entered into the device 101 by a user of the system, for example by typing an address.

The Interface for Third Party 103 is an optional component that exposes population level information 111 about a possible anomaly to a consented third party, such as an employer or a government body in charge of epidemic management, which can provide services to users of the system presented here, based on individual level information 112. In the case of an employer, this could be to require the employee to work from home on days of exposure, or to work from home on some days to achieve a certain density of individuals that are known to not be exposed or possibly infected based on predictions from the system. In some embodiments, this interface 103 is a web-site that can be displayed in an internet browser on a laptop computer, which visualizes the disease cases predicted by the system at a geographic scale. Many other embodiments are possible for communicating this information to said third party through devices that can connect to the internet and display results. A module is also specified 114 for communicating between the Third party 103 in some embodiments, where for example, the Third party 103 might be a primary care provider that is consented by a user of the system to share their medical record, or updates thereto with the epidemiology monitoring network 102 to improve accuracy of for the user or the system overall for novel epidemic discovery.

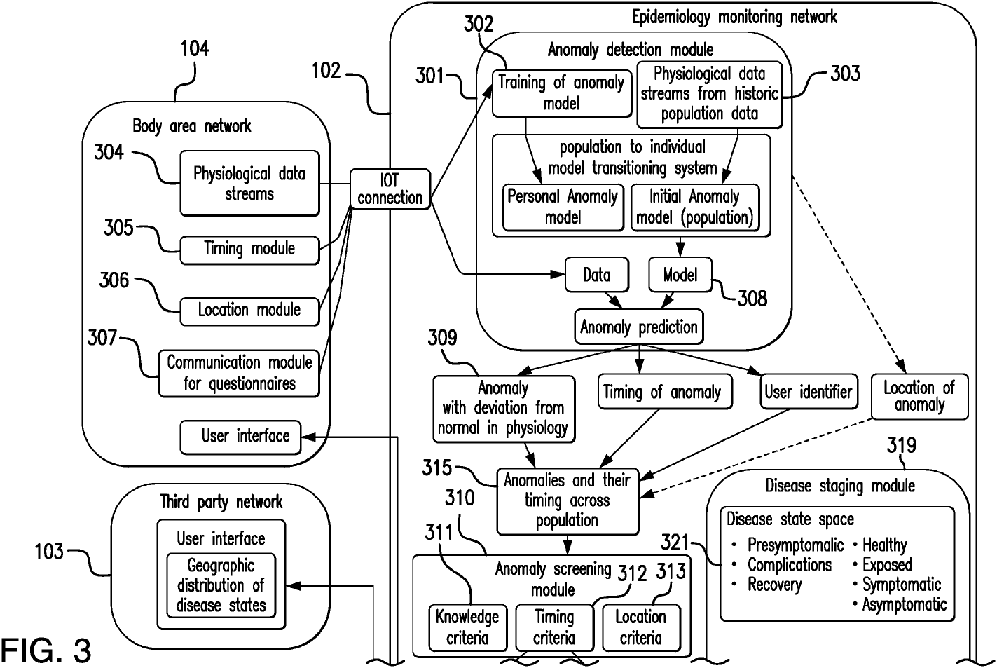

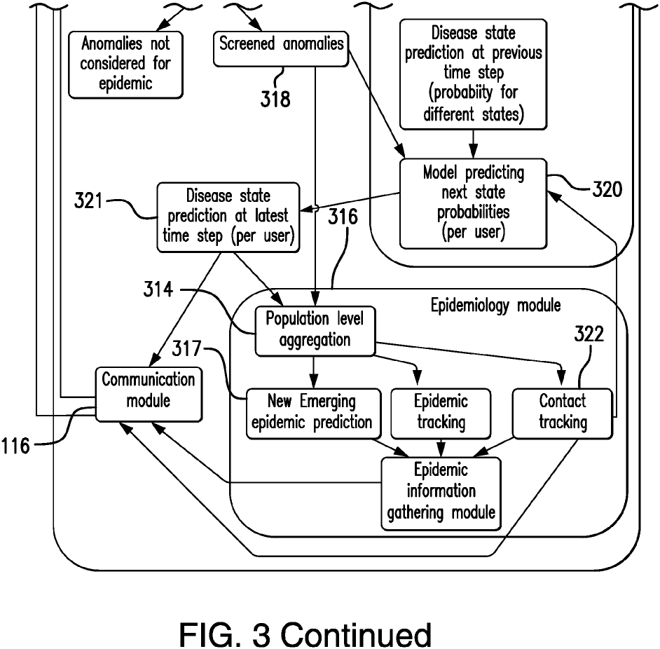

The interface to users 109, either through Mobile Monitoring and/or the Mobile devices 100, 101 in the BAN 104, or the Interface for Third party 103, have the elements necessary to relay information that can directly benefit monitored individuals in the ways described previously, e.g. to alert them to changes in their vital signs indicative of infection. More specific examples include, but are not limited to, displaying to the individual information on their inferred disease status in case of an epidemic, displaying notifications of new potential epidemics in their location, displaying information to the individual which is relevant to their general health status prior to infection and subsequent risk of developing severe disease complications, displaying notifications regarding the potential development of severe disease complications, displaying information regarding changes to the user’s level of health under local regulations published by relevant authorities intended to slow the progress of the disease, displaying the risk and disease status of other consented members of the system such as friends, colleagues and family. The interface 109, does not only notify, share or visualize relevant information for the user, but also has the ability to perform queries that help to inform in more detail, what the most likely disease state of the user might be. A more detailed view on the purpose of this process is given in the detailed description for FIGS. 3, 4 and 5.

Furthermore, in some embodiments the interface to users 109, either through devices 100 or 101 in the BAN 104, or the Interface for Third party 103, can also relay information about a monitored individual to consented third parties who may have interest in the individual’s health state, e.g. the individual’s physician. The same information can also be relayed to other consented third parties whose own health may be affected by proximity to the monitored individual in the event they become infected, e.g. family members in the same household. These are but two non-limiting examples in which problems highlighted previously in the background section may be solved. The Epidemiology Monitoring Network 102 may also contain a Communication Module 116 for the purpose of communicating directly with monitored individuals in the system; the purpose of communication can be to query the monitored individual as to symptoms experienced, convey notifications of potential infection or exposure to infected individuals, suggest medical treatment based on detected development(s) of severe complications, etc. A specific and particularly important function of the Communication Module 116 is to allow monitored individuals with clinically-confirmed infection to report said information into the disclosed system, eliminating any probabilistic uncertainties associated with predictions of infection based on observed changes in the physiological data; this information can also be used to reinforce the models which predict the presence of infection. Conversely, another specific and important function of the Communication Module 116 is to allow monitored individuals flagged for possible infection to be prompted for feedback on potential confounding factors not associated with infection, for example alcohol consumption, recent intense exercise, and consumption of heavy meals; certain physiological changes associated with these types of confounding factors (e.g. an increase in resting heart rate), which may otherwise be interpreted as infection symptoms without additional context.

The epidemiology monitoring network 102 can, in an embodiment, be hosted on one or multiple cloud servers running various other components of the proposed invention, e.g. the anomaly detection module 301, which is outlined in FIG. 3 and will be described in detail forthwith. In another embodiment, these various components of the Epidemiology Monitoring Network 102 can alternatively be hosted on the same devices comprising the BAN 104, or a subset thereof, as outlined in FIGS. 4 and 5, and can be computed on the same microcontrollers providing computation for the BAN 104. In yet another embodiment, various components of the Epidemiology Monitoring Network 102 may be arbitrarily spread across aforementioned cloud servers as well as devices comprising the BAN 104, in any combination. In general, the components are designed to be platform independent, and possible deployment configurations are limited only by the constraints of devices available, i.e. computational power and/or available memory.

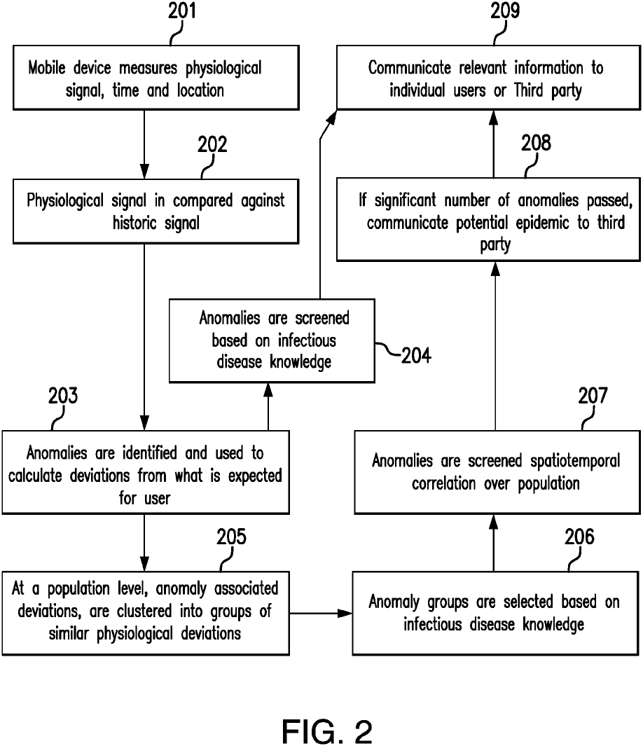

FIG. 2 describes a method 200 performed by the system according to an aspect of the present invention. In a first stage 201, physiological time series data is measured for an individual using a Mobile Monitoring Device 100. This includes many different types of physiological signal by virtue of the alternative Mobile Monitoring Device 100 embodiments already discussed, but from a physiological point of view also includes data on the five vital signs (Heart rate, breathing rate, blood pressure, body temperature and SpO2) and other basic physiological processes like sleep, sleep stages, EEG activity, EMG activity and actigraphy. The data is time-stamped by the device 100 with the appropriate time zone information in order to relate events observed on different monitored individuals in time. Time zone information can be obtained either from real-time knowledge of a monitored individual’s location when either devices 100 or 101 directly support such information (e.g. via GPS enablement), from location data entered into the epidemiology monitoring network system 102 by the monitored individual (i.e. through a user interface application on devices 100 or 101), or from location information which can otherwise be established indirectly e.g. proximity of Mobile Device 101 to cellular network towers. In one embodiment of the system, location data gathered from devices 100 or 101, or via other aforementioned means, is also included in the BAN 104 for the monitored individual allowing data in the BAN 104 to be spatially related across multiple monitored individuals.

For a given monitored individual, some or all of the continuous physiological data collected by Mobile Monitoring Device 100 is accrued and stored in any or all of the Mobile Monitoring Device 100, Mobile Device 101, or Epidemiology Monitoring Network 102. The accrued data may be “raw” data, or data which has been compressed by some reversible scheme, or statistical summaries of the “raw” data, or some combination thereof. The duration of time spanned by the accrued data, or summaries thereof, may be any interval but will generally be on the order of days to months. In the second stage 202 of FIG. 2, physiological time series data measured in the present or intermediate term (e.g. within the last 24 hours) by the Mobile Monitoring Device 100 is compared against the historical accrued data in order to identity statistically significant deviations of interest, which will hereafter be referred to as “Anomalies.” The identification of Anomalies occurs in the third stage 203 of FIG. 2. Anomalies may be identified in time series data by any applicable scheme known to those skilled in the art (https://en.wikipedia.org/wiki/Anomaly_detection). Anomalies are generally defined as rare values. This is best understood as rare with respect, to some distribution from which the data was sampled. In terms of human physiology, if we consider the parent distribution as data collected from mainly healthy individuals, data from individuals with disease or time segments of that data(events), would be rare and thereby considered as anomalies with respect to the parent distribution of mostly healthy individuals. It is also important to consider that data from an individual who moves through states of being healthy, diseased and recovered, can generate her own parent distribution during the healthy state to help flag times when the individual enters a diseased state. As a simple example, mean heart rate values measured over 24 hour periods may be aggregated on a daily basis for a monitored individual, and an Anomaly might be defined as any 24 hour mean heart rate which is removed by more than (say) 10 beats per minute from the historic norm over a span of several months. As another simple example, continuous heart rate values may be accrued into a histogram containing the statistics of all observed heart rate values of a span of several months; such a histogram can be used to obtain an empirical Cumulative Distribution Function, eCDF, for the data (https://en.wikipedia.org/wiki/Empirical_distribution_function)and an Anomaly might be defined as any subsequent 24 hour mean heart, rate value, which falls into either a very low or very high percentile of the eCDF. Such values are comparatively scarce in the overall dataset. Many particular methods are available to those skilled in the art, for defining scarce values, for example Z-scores, Interquartile Ranges, Bhattacharyya distances, etc.

Different embodiments of the disclosed invention will contain different schemes for defining and identifying Anomalies, depending on factors such as expected symptoms for diseases of interest. In an intermittent stage 204 of FIG. 2, Anomalies coincident with infectious diseases are screened and flagged based on clinical knowledge of how said diseases manifest symptomatically. For example: it is known in the clinical literature that fever is typically accompanied by an increase in resting heart rate, typically 6-8 beats per minute for every degree Celsius above norm; thus, an abnormal and acute increase in a monitored individual’s night-time average heart rate, compared against historic norms per stage 202, would suggest that the monitored individual has developed a fever and subsequently trigger a flagged Anomaly in stage 204. This information can be directly conveyed to the monitored individual and/or to consented third parties in stage 209 of FIG. 2. In stage 205 of FIG. 2, similar Anomalies from multiple monitored individuals can be clustered in the Epidemiology Monitoring Network 102, in stage 206 of FIG. 2, Anomaly groups which collectively indicate particular infectious diseases (based on established clinical knowledge) may be aggregated; for example, fever Anomalies alone may indicate any number of infectious diseases, but fever Anomalies in conjunction with Anomalously low SpO2 measurements and Anomalously high breathing rate measurements may indicate Covid-19 infection, specifically.

In stage 207 of FIG. 2, monitored individuals clustered into specific Anomaly groups may be screened for spatiotemporal proximity, which is to say that appropriately time stamped historic location data collected by the Mobile Monitoring Device 100 and/or Mobile Device 101 (e.g. GPS or location-specific network data) for the Anomalous monitored individuals may be cross-referenced in the Epidemiology Monitoring Network 102, which looks for instances in which close proximity occurred between Anomalous monitored individuals (i.e. instances in which an infectious disease may have been spread between monitored individuals). In stage 208 of FIG. 2, a sufficient number/threshold of close-proximity events identified by the Epidemiology Monitoring Network 102 may indicate an emergent or continuing epidemic, and this information may then be conveyed to affected monitored individuals (e.g. those in the vicinity of the infectious disease outbreak) and/or consented and interested third parties (e.g. public health officials) in stage 209 of FIG. 2.

As described in FIGS. 3-5, the steady recording of physiological signals by the system allows for the creation of models of the end-user’s physiology that can distinguish between typical patterns in the user’s physiology, as well as spurious patterns that have not been observed and cannot be extrapolated from historic data, dubbed anomalies 203/204. Note that FIG. 3 specifically illustrates an embodiment in which calculations supporting these functions take place predominantly on processors external to a monitored individual’s Body Area Network 104, e.g. in cloud servers which communicate with the BAN 104 via IoT communication; FIGS. 4-5 together illustrate a separate embodiment in which the same calculations occur predominantly on devices local to, or part of, a monitored individual’s BAN 104. Generally speaking, the embodiments of FIGS. 3-5 differ mainly in their implementation rather than their function.

For the most typical embodiment this process of building such models 308 using said data (see FIG. 3), will occur within the Epidemiology Monitoring Network 102, but in other embodiments this can also occur on processors and storage medium of the BAN 104. Models as described here, are effectively comparing the newly measured physiological signal in varying degrees of sophistication in different embodiments against historic physiology for said user 202. Deep learning approaches such as autoencoders, general adversarial networks, support vector machines, and others known to those skilled in the art have added new possibilities to the field of anomaly detection and modelling said recent-distribution of a system. In many instances these approaches are likely to outperform traditional methods--for example z-scores, Bhattacharyya distances, and other methods based on moment statistics -- with regards to the successful detection of Anomalies concurrent with an underlying cause (e.g. disease infection). This is particularly likely in applications where large volumes of data are available, such as in continuous body monitoring with wearable devices. One clear limitation of anomaly detection methods in general is that there is no distinction between the types of anomalies that are detected. For the present invention, based on FIG. 2, we want to leverage anomaly detection to help identify and monitor a novel epidemic across multiple users of the disclosed system. In the process outlined in FIG. 2, the anomaly detection step is applied to physiological data, yielding raw anomaly events 203, which are segments of time series data that have been flagged by an Anomaly Detection Model 308. The model 308 may, in general, be any technique or approach, or combination thereof, which can be used to screen for instances of disease infection in monitored individuals within the Epidemiology Monitoring Network 102 using measured physiological data in conjunction with other information such as, but not limited to, geospatial data, symptom reports from monitored users, known instances of disease cases within regions of interest, etc. The model 308 may incorporate mathematical or machine learning approaches for automatic Anomaly detection, e.g. the exemplary deep learning approaches or statistical approaches described previously, and will generally be programmed in such a way that Anomaly detections can be done automatically by the system. Prior to obtaining a sufficient amount of physiological data on a new monitored individual within the system, the model 308 may apply a population-based model to the new individual until such time that enough data can be accrued to build a robust individualized model.

As a first step, Anomaly associated deviations 309 in user physiology are calculated for each Anomaly 203, describing the overall character of the observation, one example being an acute increase in resting heart rate indicative of the onset of fever, as discussed previously above. These deviations can, in one embodiment, simply be the difference between the anomalous data segment and the mean values for various physiological measurements considered (e.g. per the example above); in other embodiments, it can be a more sophisticated calculation making use of the detailed distribution calculated for the user 308. Such an anomaly model 308, whether it is simply an average value of the most recent data, a probabilistic distribution or an encoder loss for a more sophisticated method such as a general adversarial network or variational autoencoder, is continuously and automatically updated in stage 302 using new wearable data from the user 304. This process of continuous training is commonplace in anomaly detection on time-series data.

This invention can utilize an Anomaly Screening Module 310, which comprises multiple screening layers: knowledge criteria 311, timing criteria 312, and location criteria 313. These layers comprise the functional implementations of stages 206 and 207 as outlined in the flow diagram of FIG. 2. These layers in conjunction increase the likelihood of detecting anomalous deviations in collected data which are most relevant to the outbreak or progression of a new epidemic. The layers do not necessarily have to be applied in the order specified and embodiments with many different permutations are possible.

In an aspect, in order to detect infectious disease outbreaks within a population, as well as infections within specific monitored individuals, all anomaly associated deviations observed in a recent time-window for a monitored population within a region of interest (e.g. family unit, workspace, city, or state) are clustered into a set of groups by unsupervised machine learning methods 205, for example K-means clustering, Gaussian mixture modeling, or other similar approaches known to those skilled in the art. These clusters can each be considered a potential disease associated cluster.

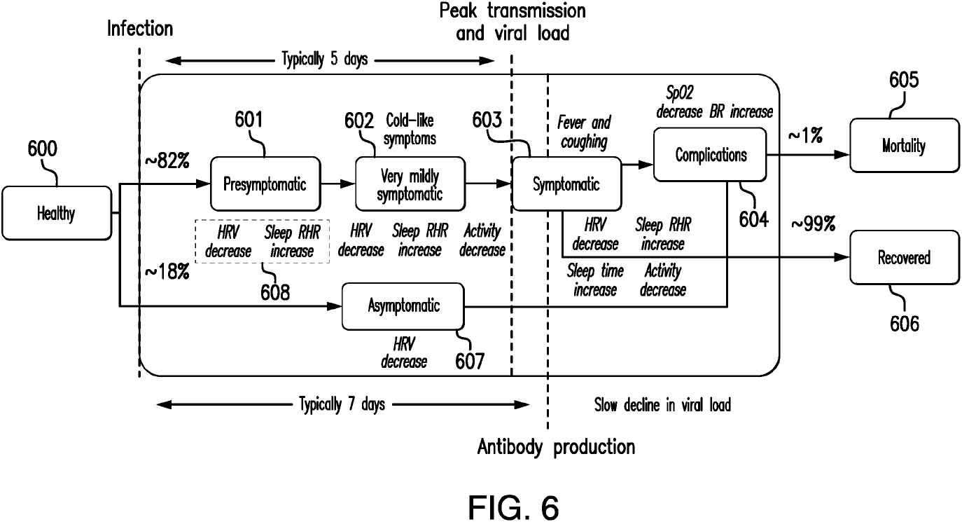

The abstract stage 206 in FIG. 2, and its associated functional implementation 311 in FIG. 3, are knowledge filters which are applied to potential disease associated clusters identified in stages 205 and 315. The knowledge filters select Anomaly associated deviations in line with what is expected from the scientific literature on how various infectious diseases manifest within populations, as well as in the vital signs of single infected individuals. How these diseases manifest, can be captured with a basic set of epidemiological parameters described in the disease state space (FIG. 6), such as the incubation period of the disease, the vital signs affected at each stage of the disease and the transmissi bility of the disease. Spatiotemporal data on Anomalies arising in vital signs during an epidemic, provide vital information on the nature of the infectious agent and can be cross-referenced against a database of known epidemiological parameters of diseases to arrive at a subset of plausible disease types based on the data.

A basic example of a knowledge filter would be the expected incubation time of a disease, i.e. the length of time between exposure and the appearance of initial symptoms. Incubation times vary widely between different diseases, with Covid-19 having an incubation period of several days, compared to a much slower incubation period of several weeks to months for tuberculosis. As a more specific example of the knowledge filter’s functionality: various clinical surveys of Covid-19 patients have produced prevalence rates for fever symptoms associated with infection, if a detected Anomaly cluster contains indications of fever at a similar rate to what is expected, then the confidence of a Covid-19 outbreak within the Anomaly cluster is increased. In an exemplary embodiment of 206 and 311, a set of knowledge criteria could be applied to various vital signs measured within a monitored population in order to screen for transitions from a healthy state to an infected but pre-symptomatic state for a given disease type, e.g. a blood borne bacterial infection, which will typically be associated with an increase in resting heart rate, a decrease in high frequency heart rate variability, and an increase in systolic blood pressure. In another exemplary embodiment, a set of knowledge criteria could be applied to screen for transitions from a symptomatic but stable stage to a degraded stage in which severe complications are developing, e.g. in viral pneumonia for which severe complications are associated with a decrease in SpO2, an increase in breathing rate, an increase in resting heart rate, and a decrease in the high frequency component of heart rate variability.

The output of stages 206/311 is a set of Anomaly associated deviations that are indicative of a disease outbreak in populations or specific monitored individuals, termed potential disease associated Anomalies. The subsequent abstract stage 207 in FIG. 2 has its associated functional implementation in stages 312 and 313 of FIG. 3, which in conjunction cluster potential disease associated Anomalies in space and time by examining the associated (time stamped) location data. This allows for calculation of the local geographic hotspot of a new outbreak, using disease case timing and location, as known in the art of Epidemiology. This context is also critical for identifying potential clusters which could otherwise be suitably explained by causes other than the outbreak of a disease, for example elevated resting heart rates observed in monitored individuals whose collective location and physiological data suggest participation in an athletic event, e.g. a public marathon (which would also account for anomalous, acute increases in resting heart rate).

In the abstract stage 208 and its functional implementation 314, all remaining potential disease associated clusters are evaluated for statistical significance compared to a baseline model of physiological variations in a healthy population. Significance tests known to those skilled in the art, for example p-value tests, allow clusters arising from true outbreaks of disease to be more reliably separated from geospatially correlated clusters arising due to random (or spurious) natural variations observed in otherwise healthy populations.

To maintain clarity in communicating the operational steps of the invention disclosed, the process for obtaining epidemiologically relevant anomalies has been disclosed purely from the point of view of obtaining a set of potentially disease associated Anomalies above. In parallel to this process, the disclosed system also makes use of a Disease Staging Module 319 for tracking explicitly the probabilities of individual disease states as additional context for monitored individuals. This Disease Staging Module 319 contains a definition of a disease state space, which is depicted in FIG. 6 for the specific case of Covid-19. A disease state space in this context refers to the set of values, or statistical range of values, in the space of the input data which are consistent with individuals undergoing an active infection (as established by a set of true positive reference data, or by a clinical understanding of the expected symptoms, or some combination thereof); points outside these values may also be considered as defining healthy, non-infected individuals. In general, a state space representation is a formalism well known to those modelling dynamic systems in control engineering (https://en.wikipedia.org/wiki/State-space_ space, representation). In mathematics and computer science, a finite-state machine (https://en.wikepedia.org/wiki/Finite-state_machine) is formalism that describes a model that has a finite number of states and a finite number of transitions at each time step between such states.

In an aspect, disease state spaces are models that describe how an individual transitions from healthy, to exposed, to symptomatic, to recovered as a finite state machine. Only certain transitions are possible - for example, in FIG. 6 we show such a Disease State Space for Covid-19 infection. It is not possible for instance to transition from Recovered 606 to Mortality 605 in a single step. In defining the Disease State Space, we can clearly define the individual states based on clinical knowledge of a disease, as well as logical transitions between these states and the timing of how an individual is expected to transition between these states; for example, the documented incubation period for a disease provides a direct estimate on the time required to transition from an Exposed state if we modelled such, to a symptomatic state. With the disease state space defined, various statistical models can be built which describe the numerical probabilities of a person transitioning from one state to the next based on the information pertaining to that individual at hand. One well known framework to those skilled in the art, for creating such a State Transition model, is a probabilistic graphical model, with an explicit example being a Hidden Markov Model (HMM), where the disease state space defines the states that are tracked in the HMM’s state vector, as well as the possible transitions between states captured in the HMM transition matrix. In order to obtain a fully trained HMM model in this example, data on transitions in the disease state space as well as contextual information such as physiological deviations that affect said transitions, form data that can be used to train the HMM. Algorithms for training such a model with such data are available, see for example the Baum-Welch algorithm (https://en.wikipedia.org/wiki/Baum%E2%80%93Welch_algorithm).

In FIG. 6, the individual disease states are numbered 600, 601, 602, 603, 604, 605, 606 and 607. It takes as input, screened anomaly associated deviations for a user (per stages 308 and 318), which are candidates for being disease related 318 and considers these together with a current estimate for the user’s disease status 321, which can assume any of the values in the disease state space 321. Per above, the system then determines whether a state transition is likely to have occurred based on the existing probabilities for the state of the user, in conjunction with transition probabilities in line with the expected physiological changes at each stage, as established via existing knowledge of particular infectious diseases under consideration. For example, the system can utilize any known new formal mathematical frameworks for performing this computation. In one embodiment of the disclosed system, a Hidden Markov Model (HMM) is utilized, where the hidden states correspond to the disease states in FIG. 6, the transition probabilities correspond to the similarity between the knowledge based transitions (e.g. 608) in the disease state space and the observed anomaly associated deviations in physiology. As a concrete example, for a user that is initialized in the Healthy state 600, the probability of that state would be 1 on the first time-step of the model and if an anomalous physiological deviation is recorded on the next time-step of the model showing decreased heart rate variability and increased resting heart rate (particularly during sleep), the HMM calculation would determine that the most likely end state would be the Presymptomatic stage 601 by transitioning the user from Healthy to Presymptomatic. The role of this step is firstly to maintain context over a larger time window to make decisions on the disease stage status of individual users, while also serving as the basis for communicating results to the user and to third parties when enough certainty is reached on the user attaining a specific disease state transition.

The disease tracking capabilities of the system, as described above, enables the automatic detection of complication development, for example the development of severe pneumonia in the case of a Covid-19 infection. To expand on this example: starting with the monitored individual that is perhaps not aware they are developing significant complications, the system automatically detects associated Anomalous vital signs in their data at stage 309; in the case of viral pneumonia, this is commonly seen as an abnormally low value of SpO2 over the continuous span of hours of days. The disease status of this individual in the Disease Staging Module 319 is in all likelihood already indicated as symptomatic, as the system would most likely have detected other vital sign Anomalies consistent with infection such as high resting heart rate (particularly during sleep) and lowered heart rate variability.

As previously established, these Anomalous changes in vital signs are determined with knowledge of how said values typically change in the presence of infection (as can be learned in clinical studies and other references known to those skilled in the art), in conjunction with acquired knowledge of the monitored individual’s historic norms pre-infection. Recurring Anomalies over multiple days’ data will add confidence to predictions of infection and help eliminate false positives which may arise due to, for example, lifestyle behaviors e.g. alcohol consumption. Continuing with the current example, the monitored individual’s SpO2 (as determined from data collected by their Mobile Monitoring Device 100) reaching an Anomalously and dangerously low value over a sustained period of time (e.g. days) becomes detected as an anomaly event in 309; again here, the values are established as Anomalously low with clinical knowledge in conjunction with acquired knowledge of the monitored individual’s historic pre-infection norms.

In the Epidemiology Module 316, this potential case of severe complication development is compared against other confirmed or probable cases of Covid-19 infection recorded within the general system, a significant statistical deviation in the exemplary individual’s vital signs with respect to those in the wider infected population, particularly one which is sustained over a period of several days or longer, will increase the confidence that severe complications are developing in this individual. Questionnaire information sent to the monitored individual via the Communication Module 116 may confirm new symptoms consistent with the viral pneumonia, e.g. a sensation of tightness in the chest upon breathing. Further questionnaire exchanges may recommend that the user follow up with her primary care physician to immediately evaluate their current condition.

The invention as disclosed in FIG. 3 focuses on a particular embodiment where the majority of Anomaly identification and screening takes place on the Epidemiology Monitoring Network 102 manifested in a cloud server, or combination of servers. Alternative embodiments, as shown for example in FIGS. 4 and 5 jointly, can move a significant amount of the system’s functionality to the devices of the Body Area Network 104, i.e. the Mobile Monitoring Device 100 and/or Mobile Device 101, such that the systems of the disclosed invention can operate in a decentralized manner (which can reduce overhead costs associated with centralized cloud computation). In the comparatively decentralized embodiment of FIGS. 4-5, the Anomaly Detection Model 308 operates locally in equivalent form 400, but its parameters are trained/calculated on a cloud server 501 which has access to population data and other contextual information (e.g. symptom knowledge from new clinical studies) which updates regularly and is therefore best served by a centralized server, or multiple servers. In this embodiment, updated model parameters trained in stage 501 are communicated to local model copies 400 by means of IoT communication.

In addition to inferences made on physiological data regarding disease stage transitions, the system also reaches out through the Communication Module 116 to users of the system to gather information on additional symptoms, in one embodiment being through questions on symptoms such as coughing, headaches and also subjective assessments of health such as the presence of chills, coughs, fatigue, difficulty breathing and general malaise. In some embodiments, the questionnaire also includes confirmation of suffering from specific communicable disease at the time as determined by a medical professional. Such data can be employed in some embodiments to train the anomaly model 302 to recognize specific disease states in addition to normal and anomalous classes, while also being used to fix the probability of specific disease states in the disease state space model at high certainty.

In embodiments where the disclosed system has access to location information via GPS 115 or other proximity measures 113, the system can also be configured to perform contact tracing 322. This is achieved by making use of recent geospatial location data for multiple users that have been within a threshold distance from an individual in the system who has either been predicted to enter a Diseased state in according to the Disease Staging Module 319, and/or who has disclosed their current status as being a confirmed positive for communicable disease using the Communication Module 116 (see FIG. 1). The process of contact tracing is defined at the time of writing as is “the process of identifying persons who may have come into contact with an infected person (“contacts”) and subsequent collection of further information about these contacts” (https://en.wikipedia.org/wiki/Contact_tracing).

Estimates of positive disease status via the Disease Staging Module 319 or disclosed disease status, combined with geospatial data for multiple individuals, provide the information required for performing the first step in contact tracing and additional information can be gleaned from follow-up questionnaires to users highlighted by the contact tracing exercise (i.e. using the Communication Module 116, or other means such as phone calls or emails). An important goal of contact tracing is to alert exposed individuals as to their risk of developing a positive infection, so as to prevent further transmission in the presymptomatic stage which is known to be a crucial factor in the rapid spreading of infectious diseases such as Covid-19. It is also possible to not only take into account GPS coordinates, but also information on likely establishments at these coordinates, such that the probability of contact at for example a gym, which can might be given a default higher transmissibility between individuals compared to other establishments where patrons do not respire with the same intensity or where they do not interact with so many pieces of shared equipment when compared to, for example, pumps at a gas station, even if the same number of visitors enter and leave per time unit.

By virtue of having access to continuous physiological datastreams, the present invention also provides the ability to track markers of general health that are indicative of the risk of complication development prior to contracting an infectious agent 600, while also informing the trajectory of recovery 606 that a user of the system is experiencing. Additionally, such markers of general health are also useful to inform authorities in charge of lock-down regulations for controlling the spread of the infectious agent on the unintended negative health consequences of social isolation and closing establishments such as gyms. In certain embodiments these general health markers are based on well-known physiological signals, such as Resting Heart Rate, Resting Heart Rate during sleep, Heart rate variability, Pulse waveform and SpO2, readily obtainable from PPG capable wearable devices, as well as activity patterns and amount of exercise obtained when combined with sensors that can perform actigraphy. Additionally, predictions of derived health metrics can also serve to inform a general health prediction, for example Biological age (regression of physiological features against chronological age for a population), sleep stages and sleep duration, acute and chronic stress.

Having thus described exemplary embodiments of the present invention, it should be noted by those skilled in the art that the disclosures are exemplary only and that various other alternatives, adaptations, and modifications may be made within the scope of the present invention. Accordingly, the present invention is not limited to the specific embodiments as illustrated herein, but is only limited by the following claims.

What is claimed:

1. A method for providing continuous monitoring of a population for infectious disease, the method comprising:
a. detecting anomaly associated deviations in IOT data-streams for a monitored individual, wherein the anomaly associated deviations comprise:
i. deviations in physiological datastreams;
ii. deviations in behavioral datastreams;
iii. deviations derived from a comparison between population data and current data of the individual; or
iv. deviations derived from a comparison between historical data of the individual and current data of the individual; and
b. screening the deviations through communication with the monitored individual to predict whether they were caused by confounding factors instead of a disease.

2. The method of claim 1, wherein the screening the deviations comprises assessing factors including alcohol consumption, heavy meals close to bedtime, strenuous exercise, or psychological stress.

3. The method of claim 1, wherein after screening the deviations to confirm causes by a disease, using the anomaly associated deviations to predict transitions in a discreet disease state space for a monitored individual using a quantitative model to predict transitions.

4. The method of claim 3, wherein the discrete disease state space includes general health to describe general disease.

5. The method of claim 4, wherein the discrete disease state space further comprises multiple disease states to represent alternative diseases or disease groups.

6. The method of claim 5, wherein the quantitative model is used to predict transitions in the discrete disease state space based upon said anomaly associated deviations, the IOT data streams of the monitored individual, and feedback related to the monitored individual.

7. The method of claim 6, wherein:
a. the lOT data streams comprise measured behavior, estimated behavior, measured physiology, or estimated physiology; and
b. the related feedback of the monitored individual comprises symptoms, disease status based on clinical test results, exposure, or confounding factors, wherein the confounding factors comprise:
i. comprise alcohol consumption, heavy meals close to bedtime, strenuous exercise, or physiological stress.

8. The method of claim 7, further comprising using epidemiological parameters known for diseases to make transitions in the discrete disease state.

9. The method of claim 8, wherein the epidemiological parameters of the disease comprises incubation time, symptomatic disease duration, a R0 value of the disease, expected physiological deviations, expected behavioral deviations, or geospatial and temporal coordinates of the monitored individual and publically available estimates of disease prevalence at the location.

10. The method of claim 6, wherein the quantitative model is trained on population data including documented cases of disease and time of transitions in the disease state space.

11. The method of claim 1, further comprising contacting the monitored individual to report the prediction of disease.

12. The method of claim 11, wherein the report comprises informing the monitored individual of possible infection and a risk of developing complications.

13. The method of claim 1, wherein the monitored individual comprises a plurality of monitored individuals, further comprising combining anomaly associated deviations for the plurality of monitored individuals in a discreet disease state space to:
a. provide predictions on the disease status and risk of developing severe complications in case of infection for each monitored individual in order to control physical access to a work site;
b. discover new epidemics or new disease outbreak hotspots in existing pandemics; or
c. perform contact tracing to warn individuals of potential exposure.

14. A method of continuous monitoring of a population for infectious disease, the method comprising:
a. capturing, from mobile devices associated with individuals, physiological signals associated with the individuals, time information, and location information related to the mobile device,
b. comparing the captured physiological signals against historic physiological signals associated with the individuals to identify anomalies;
c. calculating anomaly associated deviations from the identified anomalies;
d. clustering the anomaly associated deviations into similar groups at a population level;
e. selecting anomaly groups based on infectious disease knowledge;
f. screening the anomaly associated deviations of the selected anomaly groups on spatiotemporal correlation over the population level; and
g. communicating potential epidemic findings if the spatiotemporal correlation meets threshold.

15. The method of claim 14, wherein after calculating anomaly associated deviations, flagging the anomaly associated deviations based on infectious disease knowledge.
